(19) United States
(12) Patent Application Publication
(10) Pub. No.: US 2007/0083210 A1
(43) Pub. Date: Apr. 12, 2007

(54) Apparatus and method for minimally invasive spine surgery

(75) Inventors: Hugh D. Hestad, Edina, MN (US); Paul F. Boschert, Minneapolis, MN (US); Robert Garryl Hudgins, Burnsville, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/228,958

(22) Filed: Sep. 16, 2005

(51) Int. Cl. A61F 5/00

(52) U.S. Cl. 606/86

(57) ABSTRACT

The present invention includes systems and methods for conducting surgical procedures in a minimally invasive manner. The invention includes a guide frame for the insertion of surgical instruments with a high degree of accuracy. For example, a guide needle may be inserted into the pedicle of the spine of a patient wherein the angle in a plane transverse to the spine and in a parasagittal plane may be accurately selected. The present invention also includes apparatuses and methods for accessing screws and anchors already implanted into the spine in a minimally invasive manner. Once the screws or anchors are accessed, the apparatus and method may be used to implant various therapeutic devices, including wires, cords, etc., between the screws and anchors.

### TECHNICAL FIELD

The present invention is related to spinal stabilization devices. More particularly, this invention relates to devices, systems, and methods for accessing the spine in a minimally invasive manner.

### BACKGROUND

The spinal column is a highly complex system of bones and connective tissues that provides support for the body and protects the delicate spinal flexible connecting member and nerves. The spinal column includes a series of vertebrae stacked one on top of the other, each vertebral body including an inner or central portion of relatively weak cancellous bone and an outer portion of relatively strong cortical bone. Situated between each vertebral body is an intervertebral disc that cushions and dampens compressive forces experienced by the spinal column. A vertebral canal containing the spinal flexible connecting member and nerves is located behind the vertebral bodies.

There are many types of spinal column disorders including ascoliosis be (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease, or trauma, such as ruptured or slipped discs, degenerative disc disease, fractured vertebra, and the like. Patients that suffer from such conditions usually experience extreme and debilitating pain as well as diminished range of motion and nerve function. These spinal pathologies may threaten the critical elements of the nervous system housed within the spinal column

These spinal pathologies limit the range and threaten the critical elements of the nervous system housed within the spinal column. A variety of systems have been disclosed in the art that achieve immobilization by implanting artificial assemblies in or on the spinal column. Lateral and anterior assemblies are coupled to the anterior portion of the spine, which is the sequence of vertebral bodies. Posterior implants generally comprise pairs of rods that are aligned along the axis with which the bones are to be disposed, and which are then attached to the spinal column by hooks coupled to the lamina or to the transverse processes, or by screws inserted through the pedicles.

One problem with surgically accessing the spine to deal with these disorders is that the skin and tissue surrounding the surgical site must be cut, removed, and/or repositioned to gain access to the location where the devices are to be installed. The cutting or repositioning of skin and tissue causes damage, scarring, and trauma. Unfortunately long recovery times may sometimes result. Minimally invasive techniques for the implantation of devices to bony areas are therefore particularly desirable. Minimally invasive techniques may reduce blood loss and anesthetic required during surgery and also reduce the post-operative pain and recovery time.

One prior art method of achieving the minimally invasive placement of a screw or other device during surgery is discussed in U.S. Pat. No. 6,596,008 to Parviz Kambin et al. Kambin determines the insertion points for guide pins used in the fixation of pedicle screws by establishing an approach path on a computer tomography image. Once the angle is determined, a guide pin is inserted by hand and hammered into the pedicle. A series of cannulas or other devices are then utilized to dilate the tissue leading to the pedicle. While the angle of insertion of the guide pin in a plane transverse to the spine is determined with a relative high degree of accuracy, the pin is still inserted freehand.

### SUMMARY

The present invention includes a device and method for guiding the insertion of screws and other devices into bones or soft tissue in a minimally invasive manner.

Another aspect of the present invention is a device and method for creation of a surgical tract through muscle tissue to attach therapeutic devices between implanted pedicle screws or to allow surgery in the disc space, facets, or interspinous process space or to help access the spinal canal.

The present invention includes an apparatus for aligning a surgical instrument in a desired orientation relative to a surgical site that includes a guide support member connected to a guide member, wherein the guide support member is configured to support the guide member and allow the guide member to move the surgical instrument relative to a part of a human patient along a transverse and parasagittal plane, a guide control member attached to the guide member, the guide control member rotationally mounted to the guide support member perpendicular to a longitudinal axis of the guide member, and a guide configured to accept and hold a surgical instrument, the guide rotationally affixed to the guide control member whereby rotation of the guide control may align the guide in a desired angle in the parasagittal plane and rotation of the guide may align the guide in a desired angle in the transverse plane.

The present invention further involves a method of selecting an insertion angle into the spine for a surgical instrument in a transverse and parasagittal plan that includes producing an image of a section of the patient's spine in a transverse plane, determining on said image a desired insertion path for the surgical instrument and measuring an angle between the insertion path and a midline through the spinous process, measuring on said image a lateral distance from the patient's midline to the point at which the insertion path intersects the skin of the patient's back, marking an insertion point on the patient's back on the transverse line at the measured lateral distance from the midline, placing a radiopaque marker above the insertion point and making an anteroposterior image, and determining a desired angle of insertion in a parasagittal plane by choosing a desired path in the parasagittal plane and measuring the angle of the desired path relative to the vertical reference.

The present invention may further include an apparatus for accessing two or more bone anchors inserted into a bone of a patient that includes two or more docking posts, the docking posts being a substantially straight shaft with a first end and a second end, the first end for removable attachment to the bone anchors, an alignment tube removably and operatively attached to the second end of one or more of the docking posts, the alignment tube of a predetermined length relative to the docking post, and a locator, the locator attached to an end of the alignment tube whereby the alignment tube is aligned with the first end of a first docking post, the locator including a lumen for accepting and guiding a surgical instrument to the first bone anchor.

The present invention further includes an apparatus for attaching a therapeutic device between a set of pedicle screws inserted into the spine of a patient including two or more docking posts, each docking post being a substantially straight shaft with a second end and a first end and including a lumen through the shaft, the first end of the docking posts removably attachable to the pedicle screws inserted into the patient, the distal end including means for mating with a bore in each of the pedicle screws, the docking posts further including markings for aid in aligning the bores of the pedicle screws such that the therapeutic devices are inserted through the lumen of the docking posts and be secured between the pedicle screws.

While multiple embodiments are disclosed, still other embodiments of the present invention will become apparent to those skilled in the art from the following detailed description, which shows and describes illustrative embodiments of the invention. As will be realized, the invention may be capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a perspective view of the present invention surgical guide apparatus.

FIG. 1B is an end plan view of the surgical apparatus of FIG. 1A.

FIG. 2 is a plan view of a transverse computed tomography view of a spine.

FIG. 3A is a plan view of a lateral computed tomography or intraoperative c-arm view of a spine.

FIG. 3B is an illustration of how FIG. 2 is used to determine the angle for insertion of the surgical apparatuses in the transverse plane.

FIG. 3C shows a plan shadow view of a patient's back with a midline and surgical entry point drawn thereon.

FIG. 4A is a perspective view of a surgical apparatus for accessing bone anchors in a minimally invasive manner.

FIG. 4B is a side plan view of the interaction of the pedicle screw and the docking posts of FIG. 4A.

FIG. 5 is a side view of another embodiment of the surgical apparatus FIG. 4.

FIG. 6 is a perspective view of another embodiment of the surgical apparatus FIG. 4A.

FIG. 7A is a side view of an obturator being inserted over a guide wire during the use of the surgical apparatus of FIG. 4A.

FIG. 7B is a plan shadow view of the tip of the obturator at FIG. 7.

FIG. 8A is a side view of another embodiment of the surgical apparatus FIG. 4A.

FIG. 8B is a side shadow view of the interaction of a lateral portal with a lumen of a pedicle screw for the surgical apparatus of FIG. 8A.

FIG. 9A is a side view of another embodiment of the surgical apparatus of FIG. 4A.

FIG. 9B is a side shadow view showing the interaction of a docking post with a lumen of a pedicle screw using the surgical apparatus of FIG. 9A.

FIG. 10 is a side shadow view of the insertion of a rod through a lateral portal with a cornucopia shape.

FIG. 11 is a plan shadow view of a balloon catheter inflated between a pair of pedicle screws using the surgical apparatus of FIG. 9A.

FIG. 12A is a perspective view of another embodiment of the present invention surgical apparatus.

FIG. 12B is a top plan view of the embodiment illustrated in FIG. 12A.

FIG. 13 is a perspective view of another alternative embodiment of the present invention.

### DETAILED DESCRIPTION

Several embodiments of the present invention will now be described in view of the attached figures. It should be understood that no limitation on the scope of the invention is intended. Any alterations or further applications of the principles of the invention that would normally be made by one of skill in the art to which the invention relates are contemplated. Unless specified otherwise, any known materials may be used to construct the structures disclosed herein.

The present invention apparatuses and methods are directed to minimally invasive spinal surgeries. One aspect may be an apparatus and method for the insertion of anchors, screws, needles, etc. into a bone. The invention helps to stereographically establish insertion trajectory angles for surgical access instruments in both a transverse and a parasagittal plane (when used in procedures related to the spine). The transverse plane is a vertical plane that cuts laterally across the spine of the patient. The parasagittal plane is a vertical plane that runs substantially parallel and slightly offset from the spine. In addition, the apparatus guides the insertion of a selected surgical instrument. Such a technique may simplify percutaneous techniques, decrease muscle injury, decrease recovery time, and eliminate having to approximate insertion angles.

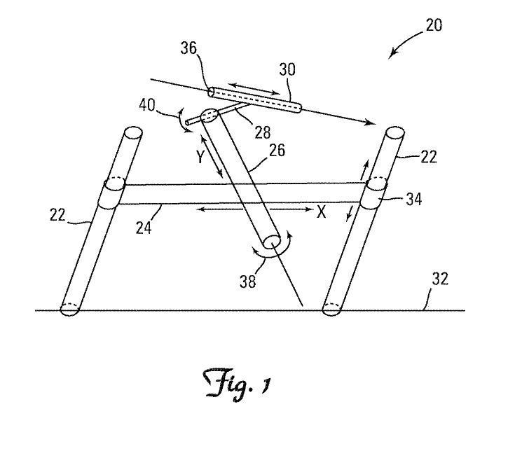

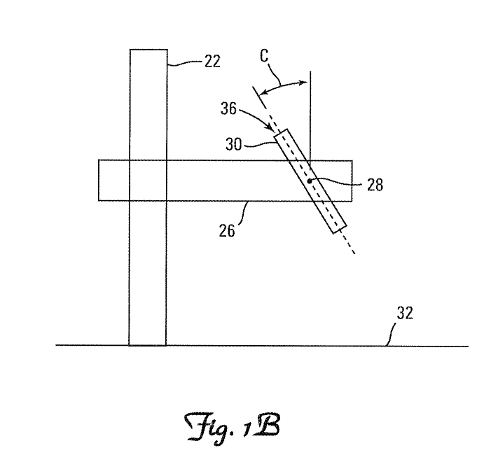

FIGS. 1A-B illustrate, one embodiment of the present invention instrument for insertion of a surgical instrument into a patient and includes guide frame 20. Guide frame 20 may include a pair of vertical supports 22, a crosspiece 24, a cantilever arm 26, a guide arm lever 28, and a guide arm 30. The guide frame 20 may be removably or permanently attached to a table 32 by attaching the vertical supports 22 at a desired location. The crosspiece 24 (also known as a guide control member) may be attached at both ends to the vertical supports 22 (also known as a guide support members) such that the crosspiece 24 may be selectively moved to a higher or lower position as desired. The cantilever arm 26 (also known as a guide member) may be movably and rotatably attached to the crosspiece 24 in a position substantially perpendicular to vertical supports 22. The cantilever arm 26 may be adjusted in both the X and Y directions relative to the crosspiece 24 and also rotated about its own longitudinal axis 38. The guide arm lever 28 may be rotationally affixed to a portion of the cantilever arm 26 perpendicular to the longitudinal axis 38 of the cantilever arm 26. The guide arm lever 28 may be rotationally mounted so that the guide arm lever 28 may be rotated around a longitudinal axis 40. The guide arm 30 (guide) also may be attached to the distal end of the guide arm lever 28. In one alternative embodiment there may only be one vertical support 22. Moreover, the vertical support 22 may be perpendicular to the surgical table but may also be oriented relative to the patient.

The guide arm 30 may include a lumen 36 through its longitudinal axis. The lumen 36 may be a hollow bore that runs all the way or substantially all the way through the entire guide arm 30. The lumen 36 may have a variety of widths depending on the application for which the guide frame 20 will be utilized. In one embodiment, guide arm 30 may be detached from the guide arm lever 28 so that a guide arm 30 of a desired length and lumen width may be selected. Alternatively, different tubes of different diameters may be placed inside of the lumen 36 to adjust the diameter and/or length of the lumen 36 to allow the guide arm 30 to accommodate different surgical instruments.

The pieces of the guide frame 20 may be attached to each other in any way known to those skilled in the art such as with clamps, screws, clasps, locks, braces, rivets, couplings, links, etc. As may be appreciated, the level of sophistication of the connection of the various pieces of the guide frame 20, including the manner in which the pieces are mounted, moved, and locked, does not affect the scope of the present invention.

The crosspiece 24 may be movably mounted to the vertical supports 22 such that the height of the crosspiece 24 relative to the table 32 may be selected and then locked. In a simple embodiment, the vertical supports 22 may include clamps that slide up and down the vertical supports 22 to a desired height and which are then locked with a thumbscrew. In other embodiments, the crosspiece 24 may move up and down the vertical supports 22 on a geared track or may be motorized.

Rotationally mounting the cantilever arm 26 relative to the crosspiece 24 allows the guide arm 30 to be moved in a parasagittal plane relative to the spine. Rotation of the guide arm lever 28 allows for movement of the guide arm 30 in a transverse plane relative to the spine. Alternatively, or additionally, the guide arm 30 may be mounted to the guide arm lever 28 to provide for selection of a trajectory in the parasagittal plane or transverse plane. Both the cantilever arm 26 and the guide arm lever 28 are preferably lockable after the desired angle has been selected. The angle of the cantilever arm 26 may be selected relative to the crosspiece 22 by sight adjustment utilizing markings on the crosspiece 22 or by some more sophisticated selection apparatus, such as a digital display. The same may be true for selecting the angle of the guide arm lever 28.

In one method of using the guide frame 20, insertion of surgical instruments with a high degree of accuracy may be accomplished. A surgical trajectory may be determined from a combination of computed tomography views and intraoperative fluoroscope fluoroscopy using both anterior-posterior and lateral views. A method of determining the insertion trajectory in the transverse plane may be substantially discussed in the U.S. Pat. No. 6,596,008 patent to Kambin “Kambin”), which is incorporated by reference for all it teaches and discloses. But unlike the patent to Kambin, the present invention additionally allows for selecting a trajectory in the parasagittal plane and for accurate insertion along the selected trajectory. Kambin avoids the need for selecting a trajectory in the parasagittal plane by putting the patient in a “flat back” position. Moreover, Kambin only contemplates free hand insertion after the trajectory is determined.

In one embodiment, the present invention method provides for the insertion of a piercing needle followed by the insertion of pedicle screws. As may be appreciated, any number of surgical instruments may be inserted or a surgical area accessed using guide frame 20.

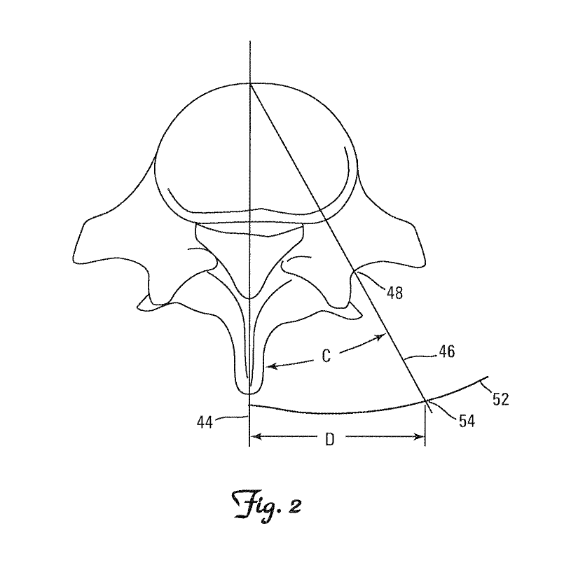

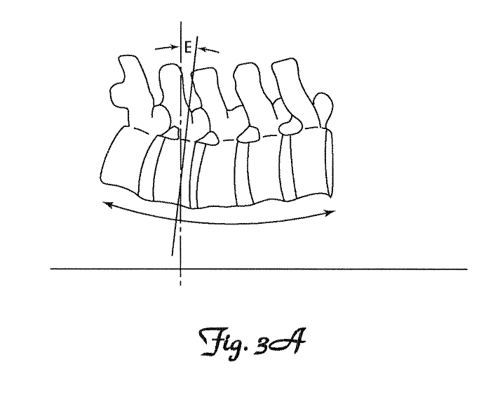

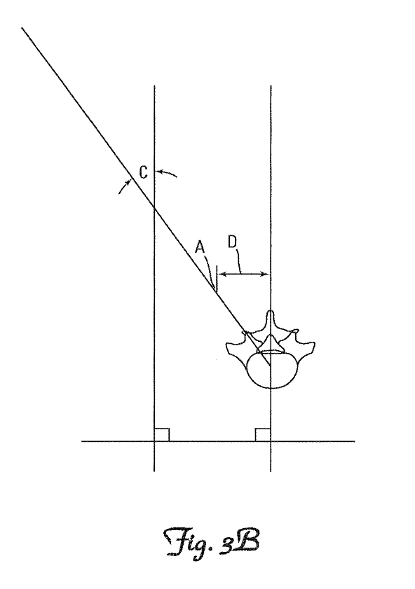

An axial scan through the pedicle center may be first taken. Such a view may be illustrated in FIG. 2. A different view may be taken for each spinal level where surgery may be to be performed. On the image a midline 44 may be drawn through the midline of the vertebral body and the spinous process. A screw path line 46 may be then drawn along the desired insertion path. The screw path line 46 should go through a desired pedicle entry point 48 and intersect the midline 50 and the skin surface 52. An angle C may then be measured between the midline 44 and the screw path line 46 by using a protractor or other measuring device. The angle C may be the trajectory in the plane transverse to the spine. A measurement may also be taken to determine the distance D between the midline and the intersection of the screw path line 46 and the skin surface to find a skin entry point 54. As shown in FIG. 3B, the pedicle trajectory angle C may be the angle the screw should be inserted at distance D from the midline (the skin entry point 54).

As shown in FIG. 3C, a grid may be made on the patient's back showing the location of a plane 45 through the midline 44. Transverse lines 53 may be drawn through the center of the pedicles intersecting the midline plane 45 at approximately a right angle. A distance D may be taken from the midline 44 laterally along the transverse lines to determine the skin entry point 54. The guide frame 20 may be then mounted on the table 32 (if not already mounted) and adjusted so that the vertical supports 22 are aligned parallel to the patient's midsagittal plane. As illustrated in FIG. 1B, the angle C for the guide arm 30 in the transverse plane may be selected by twisting the guide arm lever 28. The guide arm lever 28 may be also at angle C relative to the vertical supports 22. The distal end of the guide arm 30 may then positioned at the skin entry point 54 by moving the cantilever arm 26 and the crosspiece 24 to adjust the position of the guide arm 30 in the vertical and horizontal planes.

A fluoroscope image may then be obtained as illustrated in FIG. 3A and the insertion angle E in the parasagittal plane may be determined by measuring the fluoroscope image with a protractor or other similar device or by using an alignment arm provided on the apparatus. When utilizing such a method to determine the angle in the parasagittal plane, the guide arm 30 should be made of a radiopaque material so that the proper trajectory is more easily identified against the spinal structures in the fluoroscope image. The guide arm 30 may then be aligned with the desired angle in the parasagittal plane by adjusting the guide arm 30 relative to the guide arm lever 28. In an alternative embodiment of the guide frame 20, a radiopaque vertical guide center may be attached to a distal end of the cantilever arm 26 to aid in the selection of the angle in the parasagittal plane easier.

The pedicle may then be accessed by first slightly raising the guide arm 30 from the skin entry point 54 and making a small incision centrally over the skin entry point 54. The incision may be preferably about 1 cm long, but may be longer or shorter depending on the requirements of the particular procedure. The guide arm 30 may then be lowered back to the skin entry point 54, being careful to retain the same selected angles in the transverse or parasagittal planes. Next, a needle guide sleeve of a selected size may be loaded into the guide arm 30 and a guide needle may be advanced through the fascia and musculature until it contacts the desired point on the pedicle. The guide needle position may be verified with the fluoroscope. The guide needle may be tapped into the bone to create a pilot hole for the pedicle screw.

The needle guide sleeve may then be removed and replaced with a larger diameter guide sleeve. Larger and larger guide sleeves (also known as dilators) may be successively placed over the guide needle to dilate the tissue. The last dilation step may be accomplished by placing a working cannula over the guide sleeve and guide needle. The guide needle and guide sleeve may then be removed, leaving the working cannula in place. The pedicle screws or other bone anchor may now be placed through the cannula. Alternatively, the pedicle screws or bone anchors may be designed with a lumen so as to pass over the guide needle so that the guide needle does not need to be removed prior to insertion of the pedicle screws or bone anchors. Furthermore, if the surgeon prefers to manually retract the tissue to the pedicle, the guide needle may be used as a tract for blunt dissecting instruments and a retractor.

The guide frame 20 may be moved away from the surgical site once the tissue is dilated. The guide frame 20 may also be kept close to the operative site so that the guide arm 30 may be used as a visual reference for the trajectory of the pedicle screw during insertion. The frame can also act to hold a working cannula in a desired position.

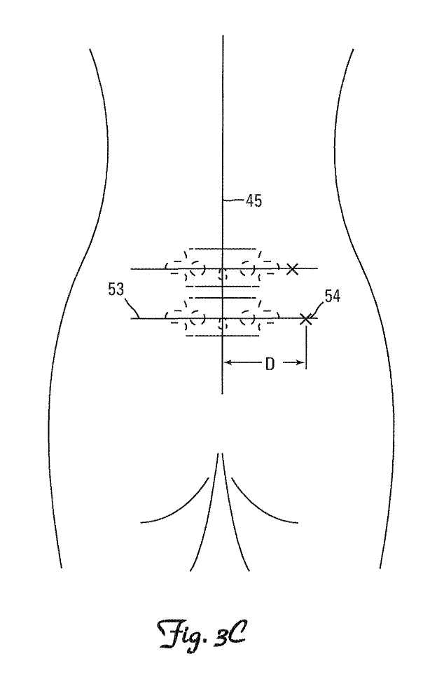

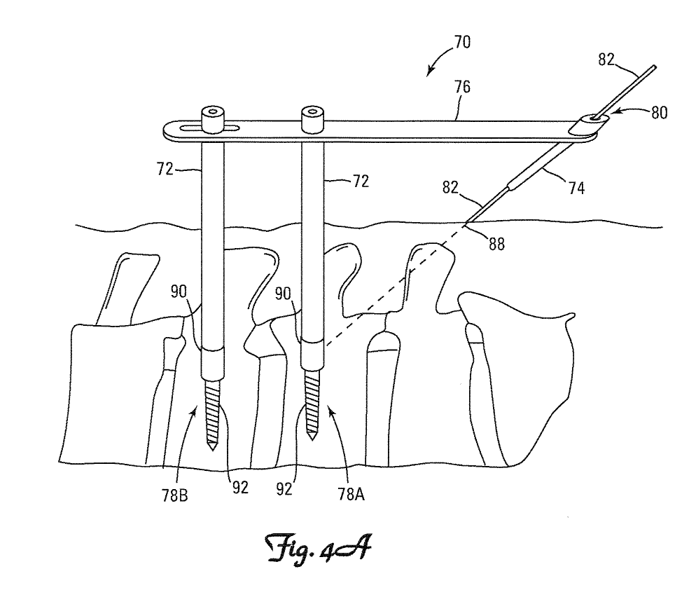

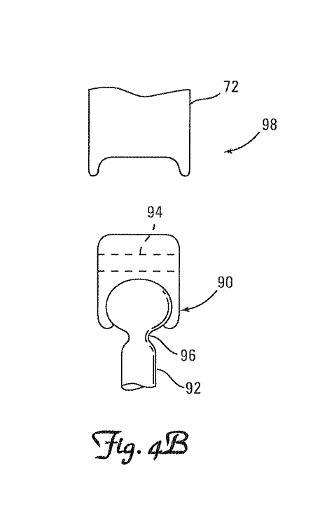

In one alternative embodiment of using guide frame 20, a working zone as defined in Kambin et al., “Arthroscopic Microdiscectomy: An Alternative to Open Disc Surgery.” The Mount Sinai Journal of Medicine, 67:4, 283-287, September 2000 “Kambin II”), which is incorporated herein by reference for all it teaches and discloses, may be accessed. As illustrated in FIG. 4 of Kambin II, a working zone for accessing the disc space may be defined by the posterior margin of the nerve root, the superior endplate of the inferior vertebral body, and the lateral margins of the facet joint. The same or similar zone may be identified as the foraminal annular window by Yeung et al., “Posterolateral Endoscopic Excision for Lumbar Disc Herniation.” Spine. 27:7, 722-731 2002, which is incorporated herein by reference for all it teaches and discloses.

Some methods of utilizing the guide frame 20 may include, but are not limited to, arthroscopic nuclectomy and nucleoplasty, arthroscopic fragmentectomy, and the placement of facet screws and in-situ curable spinous process spacers. Those of skill in the art may also realize additional methods of utilizing the guide frame 20 to assist in minimally invasive surgical techniques. Moreover, the guide frame 20 may be used for non-minimally invasive procedures, i.e. open procedures, where an accurate trajectory may be desired. Such an application where an accurate trajectory may be desired may be during surgery through the medullary canal of the pedicle.

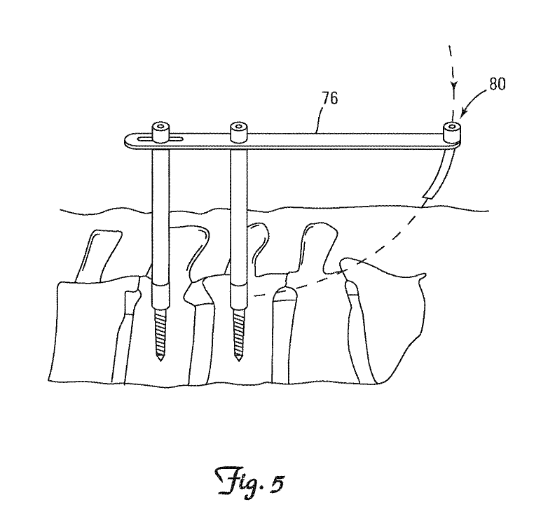

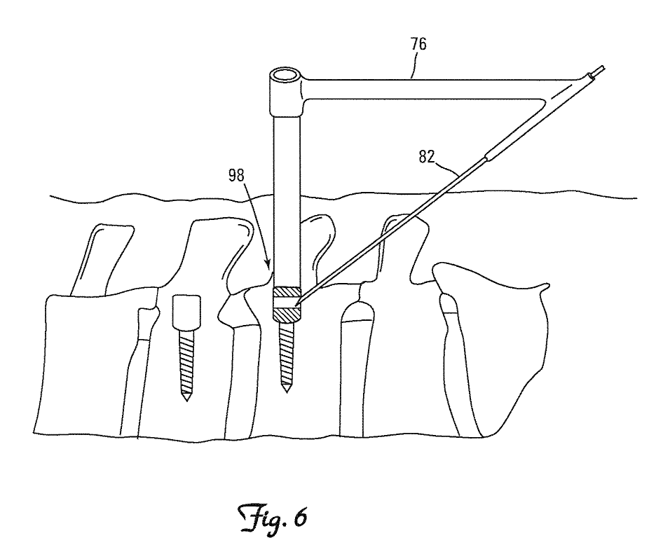

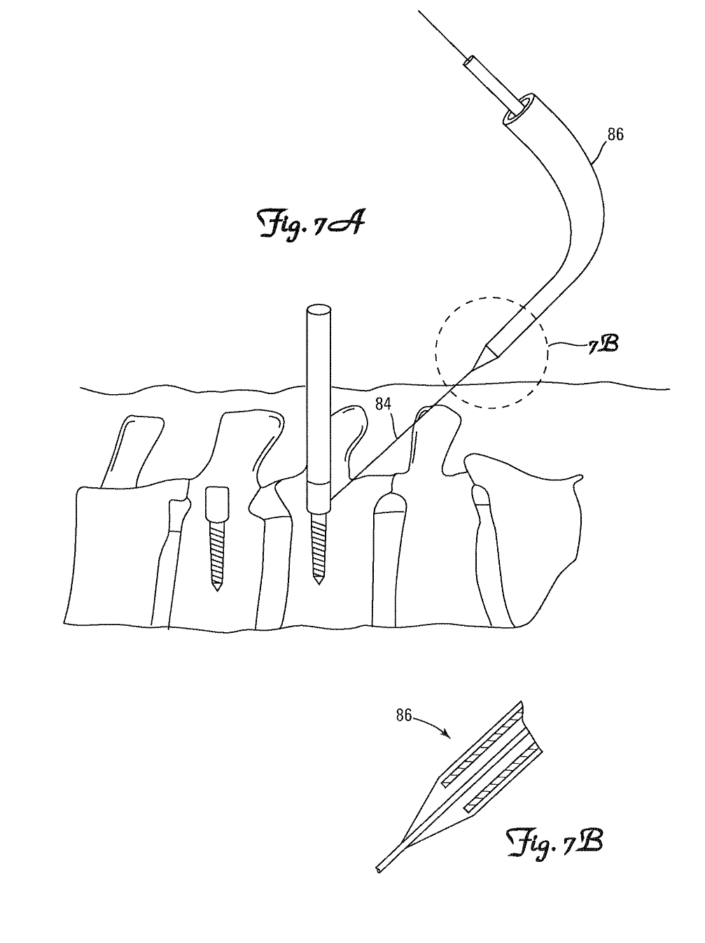

In another aspect of the present invention a minimally invasive system 70 for accessing implanted pedicle screws or other bone anchors to attach various instruments and structures between them will be described in view of FIGS. 4-13. The system 70 may be used to create a dilated surgical tract that allows attachment of therapeutic devices between bone anchors 78A and 78B engaged to bony parts of the body in a minimally invasive manner. Therapeutic devices may include wires, rods, cords or other means of connecting bone anchors 78A and 78B inserted into the spine. The system 70 allows for a minimally invasive technique of accessing the bone anchors 78A and 78B to reduce the cutting of muscles, to reduce scar tissue following the surgery, and to reduce the recovery time after surgery. The system 70 may also be used as part of an overall method of performing minimally invasive surgery that includes inserting bone anchors 78A and 78B with the guide frame 20.

The pedicle surgery system 70 includes one or more docking posts 72, an alignment tube 74, and a locator 76. The docking posts 72 attach at a first end to the bone anchors 78A and 78B. The locator 76 attaches to a second end of the docking posts 72 and the alignment tube 74 attaches at one end of the locator 76.

The embodiment herein illustrated may be described in terms of pedicle screws 78A and 78B as the bone anchors 78A and 78B, however, any surgical implant may be accessed and connected utilizing the present invention. As shown in FIG. 4B, the pedicle screws 78A and 78B of the present embodiment include a head 90, screw shaft 92, and a bore 94. The head 90 includes the head of the screw and any polyaxial means attached thereto. The head 90 may be attached to the screw shaft 92 through a connection 96. The bore 94 may be through the head 90 of the shaft and may be designed for receiving the desired therapeutic device. The head 90 of the screws 78A and 78B may include a tool opening, such as a hex, phillips, or the like, that may be configured to receive a driving tool and also include a yoke pivotally attached to the head. Pedicle screws 78A and 78B may include threads formed on the shaft 92 to engage the bone. In one embodiment, the pedicle screws 78A and 78B engaged by the present invention may include a poly-axial screw assembly. The poly-axial screw assembly may have a specialized head permanently or removably attached to the screw shaft 92 and may receive a specialized tool for driving the screw shaft into the bone. Furthermore, the pedicle screws 78A and 78B utilized in the pedicle surgery system 70 may be pedicle screws utilized in systems like the Dynesys™ system. Such pedicle screws 78A and 78B may include a hole or lumen through which a cord, cable, rod, or other device may be placed.

As may be known in the art, the head 90 can assume a plurality of angles relative to the screw shaft 92. The bore 94 (or lumen) may allow the screws 78A and 78B to receive the therapeutic device. The bore 94 may go through the head 90, shaft 92, connector 96, or some combination. Various types of bone anchors, pedicle screws, or poly-axial pedicle screw assemblies can be engaged. The pedicle screws 78A and 78B may have been placed by any method known to those in the art or by using the guide frame 20 described above.

The docking posts 72 may include a screw engaging member 98 attached on the first end thereof. The screw-engaging member 98 may be of a size and shape to engage some portion of the pedicle screws 78A and 78B. In one preferred embodiment the screw-engaging member 98 may be specifically formed to engage the head 90 of the pedicle screws 78A and 78B and may include a screw-engaging member 98 to engage the pedicle screws 78A and 78B at a known orientation. Such a specific orientation fitting system may be used to align the bores 94 of the pedicle screws 78A and 78B at a desired orientation.

The locator 76 may be a substantially straight bar, rod, or member that attaches to a second end of the docking posts 72 and extends some lateral distance beyond one or both of the posts. The locator 76 may also be curved, bent, and/or malleable in order to conform to varying spinal anatomies and lordic curvatures. The locator 76 may be attached to the docking posts by a screw, snap, compression fitting, or any other releasably locking mechanism known to those in the art. The locator 76 may include various cutouts, shapes, or other fittings to aid in fitting the ends of the alignment tubes 72. The docking posts 72 may be used to rotate the pedicle screws 78A and 78B in order to align each respective bore 94 such that the desired therapeutic device may be placed from one pedicle screw 78A to the next pedicle screw 78B. In addition, in one embodiment, the alignment tube 74 and locator 76 may have a shaped connection fitting so as to insure that the bores 94 of screws 92 are properly aligned to receive the medical device to be placed between them. Such a shaped connection fitting may include a key fitting.

The alignment tube 74 may be attached to one end of the locator 76. The alignment tube 74 may include a lumen 80 along an interior longitudinal length. The docking posts 72, alignment tube 74, and the locator 76 may be of such a length, and the alignment tube 74 may be attached to the locator 76 at such an angle, that the alignment tube 74 points along a straight line to the head of the pedicle screw 78A. As may be appreciated, the angle of the alignment tube 74 to the locator 76 may depend on the height of the docking posts 72 and the length of the locator 76 and the alignment tube 74.

In operation, the docking posts 72 may be first connected to the pedicle screws 78A and 78B. The docking posts 72 may be inserted through the back of the patient down the opening through which the pedicle screws 92 were inserted into the spine. In other embodiments, when finding pedicle screws 92 that have been previously inserted, a piercing needle may be utilized to guide the docking post 72 to the head of the pedicle screws 78A and 78B. The docking posts 72 engage the pedicle screws 78A and 78B. The pedicle screws 78A and 78B may then be rotated to achieve alignment of the bores 94 of the pedicle screws 78A and 78B.

As illustrated in FIGS. 4-7, the alignment tube 74 may be attached across the second ends of the docking posts 72. The locator 76 may then be attached to one end of the alignment tube 74. Where the locator 76 may be attached will determine which pedicle screw 78A and 78B will be accessed. The locator 76 may identify a path 88 to the first pedicle screw 78A.

A piercing needle 82 may be placed through the locator 76 percutaneously to the pedicle screw 78A and a guide wire 84 may be inserted. Once the guide wire 84 may be inserted, a lateral portal 86 may be installed over the guide wire 84. The lateral portal 86 may have an obturator tapered to feed the lateral portal 86 through the tissue without tearing or cutting. Once the lateral portal 86 and obturator reach the pedicle screw 78A, the obturator may be pulled out of the lateral portal 86. The lateral portal 86 may then be locked onto a head 90 of the pedicle screw 78A using a set screw (not shown) or other mechanism.

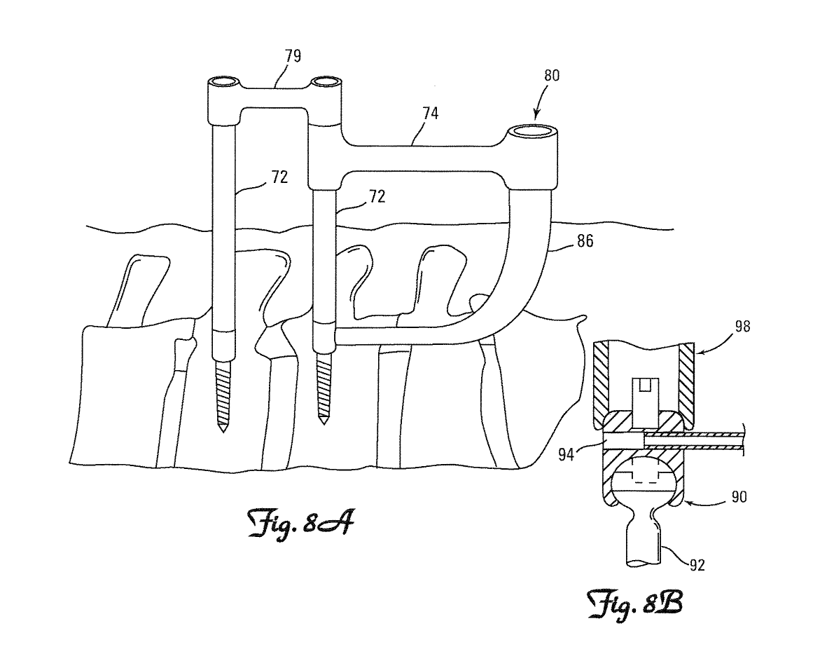

In another embodiment illustrated in FIG. 8A, docking posts 72 may be connected to pedicle screws 78A and 78B. The alignment tube 74 may be attached to one of the docking posts 72 and a connector 79 may be fitted between the docking posts 72. The connector 79 may be of such a length and fits on the docking posts 72 at a pre-selected orientation such that, because the docking posts 72 fit on the pedicle screws 78A and 78B in a particular orientation, the connector 79 aligns the bores 94 of the pedicle screws 78A and 78B. With the bores 94 properly aligned the alignment tube 74 may be utilized with the lateral portal 86 to engage the head of the pedicle screw 78A. The desired surgical instrument, such as a piercing needle 82, may then be placed down the lumen 80 of the portal 86. In further embodiments, the connector 79 may include a means for varying the length and a locking system to select a certain length. In still further embodiments the connector 79 may be in two engageable pieces. Since not all patients will be in a flat back position as illustrated in FIG. 8A, one piece of the connector 79 may be attached to each docking post 72 and the patient's spine may be adjusted until each end of the connector 79 engages the other, bringing the bores 94 of the pedicle screws 78A and 78B into proper alignment and orienting the alignment tube 74 into the proper position. Such a locking system may be utilized with any of the embodiments described herein.

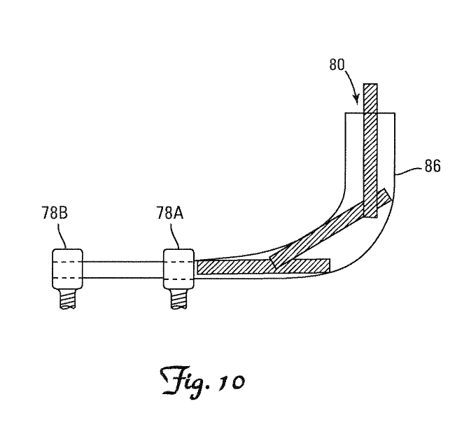

In one embodiment the lateral portal 86 may be straight. In another embodiment the lateral port 86 may be a curved or cornucopia shape. One embodiment illustrated in FIG. 10 may include a larger diameter end closest to the operator with a continuously changing radius that transitions and tapers to a straight tip. Such a variable radius lateral portal 86 may guide straight rods or other therapeutic devices through the bore 94 of pedicle screw 78A. Utilizing a curved shape for the lateral portal 86 may allow the lateral portal 86 to be inserted closer to the docking post 72 and pedicle screw 78A for an approach that penetrates less of the patient's skin and muscle. As may be appreciated, the lateral portal 86 selected should correspond to the length of locator 76 and alignment tube 74 selected.

The insertion of the lateral portal 86 may create a trajectory from the lateral portal 86 to pedicle screw 78A and to pedicle screw 78B. A tissue tract may be established between the pedicle screws 78A and 78B by inserting a shape memory device or a nickel titanium wire or rod through the lateral portal 86 and through the bore 94 of pedicle screw 78A. Another method for creating a tissue tract may be to pass a steerable guide wire from one pedicle screw 78A to pedicle screw 78B. Still another method for creating a tissue tract may be to use a wire that has shape memory such as a Nitinol wire. Therapeutic devices for securing the pedicle screws 78A and 78B may then be inserted.

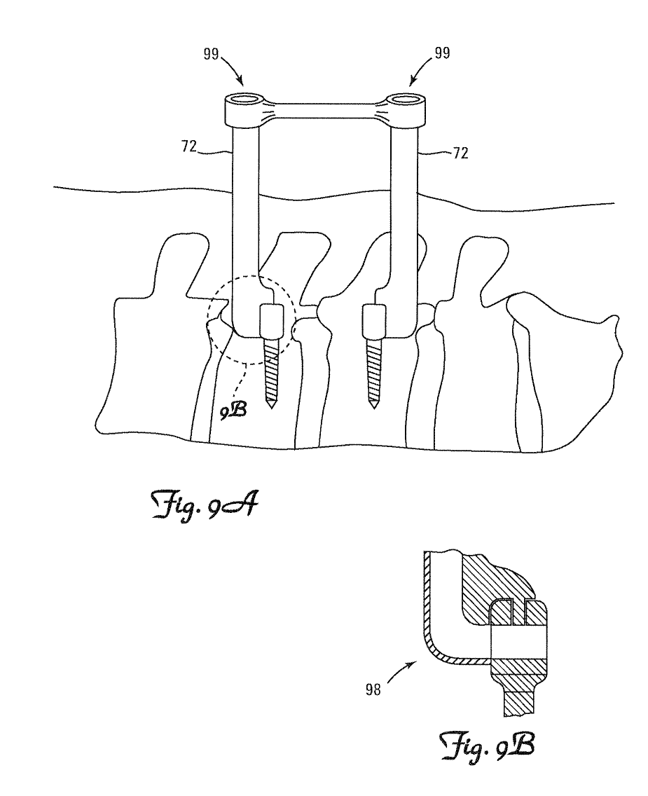

In another embodiment illustrated in FIGS. 9A-B, the docking posts 72 may include a lumen 99 for insertion of the selected therapeutic device through the docking post 72. The locator 76 may further include means for aligning the two pedicle screws 78A and 78B prior to insertion of the therapeutic device.

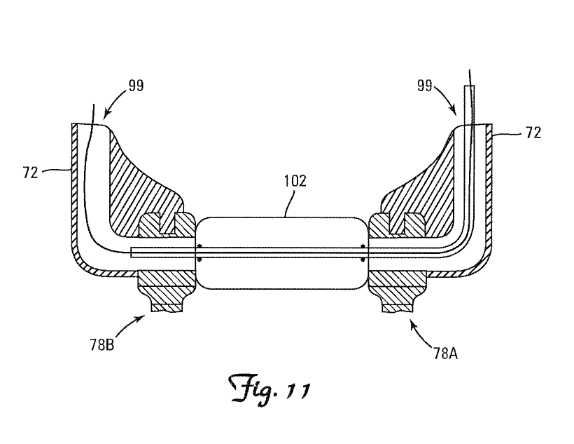

Stabilization devices may be inserted between or otherwise engaged to the pedicle screws 78A and 78B using any combination of the different embodiments of the docking posts 72 and lateral portals 86, including wires and rods made of metal or plastics, cords, or any other therapeutic device known to those in the art. As shown in FIG. 11, one such device may be a balloon catheter. The balloon catheter 102 may be inserted between the pedicle screws 78A and 78B across a guide wire and then inflated to induce blunt dissection. By continuing dilation and pressure on the tissue beyond the tissue's relaxation time the balloon catheter 102 may prevent the surgical tract from closing. The pressure on the tissue may also reduce bleeding. Blunt dissection may create sufficient sized openings to allow installation of various therapeutic devices, such as a cord, that cannot be inserted directly through the tissue.

Another stabilization device that could be inserted between the pedicle screws 78A and 78B may be an in-situ curable polymer. A second balloon catheter 102a (not shown) may be inserted into the space created using balloon catheter 102 and an in-situ curable polymer may be filled and then cured to create a cylinder of the selected elastomer between the screw heads. Once the polymer is cured, the balloon catheter 102a may be cut and the balloon, guidewire, and portals removed. Variations on such a method may be utilized, such as, for example, removing the guidewire after insertion of the balloon 102a and before insertion of the polymer. In further embodiments the guidewire may be utilized to pull a tensioning member between the pedicle screws 78A and 78B. Such methods and apparatuses may be used to implant a Dynesys™ type system in a minimally invasive manner. In one alternative embodiment the balloon catheter 102 that may be used to dilate the tissue could also be used to as a reservoir for the in-situ curable polymer instead of using two separate balloons.

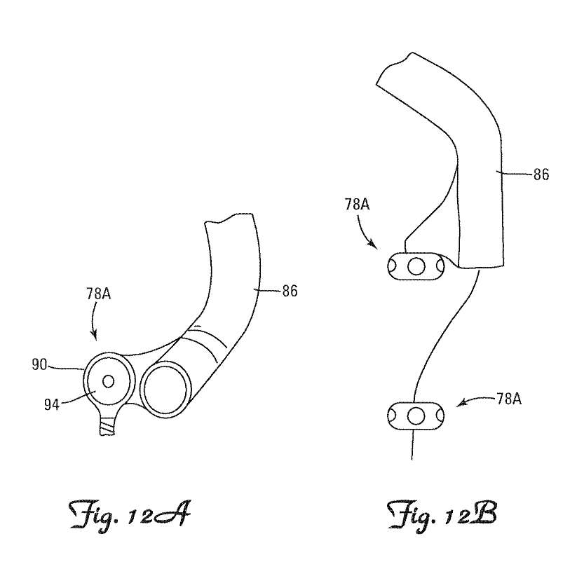

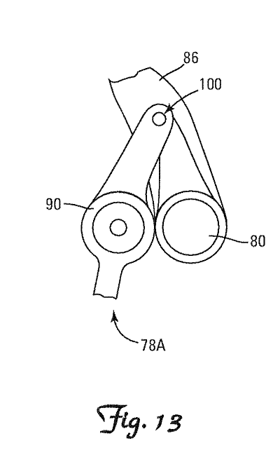

In another embodiment, illustrated in FIGS. 12A-B, the lateral portal 86 may be positioned next to the pedicle screw 78A rather than directly to pedicle screw 78A. The lateral portal 86 may be docked next to the pedicle screw 78A to allow for items to be placed between the pedicle screws 78A and 78B that would not normally be passable through the head 90 and bore 94 of the pedicle screws 78A and 78B, such as the Dynesys™ system. Such a design may include a way to dock the lateral portal 86 to the head 90 of the pedicle screw 78A and may include an offset position for the tip of the lateral portal 86. An alternative embodiment to such a lateral portal 86 and pedicle screw 78A arrangement may be shown in FIG. 13 wherein the lateral portal 86 includes a pivot point for fine adjustment of the angle for insertion of the therapeutic device.

Various modifications and additions to and various combinations of aspects of the exemplary embodiments discussed may be made without departing from the scope of the present invention. For example, where two components are noted previously to be “attached,” it may be also contemplated that, in at least some cases, they could be unitary. Accordingly, the scope of the present invention may be intended to embrace all such alternatives, modifications, combinations, and variations as fall within the scope of the claims, together with all equivalents thereof.

1. An apparatus for aligning a surgical instrument in a desired orientation relative to a surgical site, comprising:
a guide support member connected to a guide member;
a guide control member attached to the guide member, the guide control member is rotationally mounted to the guide support member generally perpendicular to a longitudinal axis of the guide member; and
a guide configured to accept and hold the surgical instrument, the guide rotationally affixed to the guide control member whereby rotation of the guide control aligns the guide in a desired angle in the parasagittal plane and rotation of the guide aligns the guide in a desired angle in the transverse plane.

2. The apparatus of claim 1 wherein the guide has a lumen configured to hold the surgical instrument.

3. The apparatus of claim 1 wherein the guide is rotationally attached to the guide control member.

4. The apparatus of claim 1 wherein the surgical instrument is a guide needle.

5. The apparatus of claim 1 further comprising means for determining the angle of the guide in a plane transverse to the surgical site.

6. The apparatus of claim 1 further comprising means for determining the angle of the guide in a plane parasagittal to the surgical site.

7. A method of selecting an insertion angle into the spine for a surgical instrument in a transverse and parasagittal plane, comprising:
producing an image of a section of the patient's spine in a transverse plane;
determining on said image a desired insertion path for the surgical instrument and measuring an angle between the insertion path and a midline through the spinous process;
measuring on said image a lateral distance from the patient's midline to the point at which the insertion path intersects the skin of the patient's back;
marking an insertion point on the patient's back on the transverse line at the measured lateral distance from the midline;
placing a radiopaque marker above the insertion point and making an anteroposterior image; and
determining a desired angle of insertion in a parasagittal plane by choosing a desired path in the parasagittal plane and measuring the angle of the desired path relative to the vertical reference.

8. An apparatus for inserting a guide needle, comprising:
at least one guide supports;
a crosspiece attached to the at least one guide supports whereby the position of the crosspiece is selected and secured;
a cantilever arm rotationally and movably mounted to the crosspiece in a position substantially perpendicular to the crosspiece, the cantilever arm mounted to the crosspiece such that cantilever arm may be positioned and secured in a desired position relative to the crosspiece;
a guide arm lever rotationally and lockably mounted to the cantilever arm;
a guide arm operatively attached to the guide arm lever, the guide arm including a lumen for accepting the guide needle.

9. The apparatus of claim 8 further comprising at least one tube insertable into the lumen of the guide arm to selectively adjust the size of the lumen through the guide arm.

10. The apparatus of claim 8 further comprising at least one means for tracking the angle of the guide arm in at least one plane.

11. An apparatus for accessing two or more bone anchors inserted into a bone of a patient, comprising;
at least two docking posts, the docking posts being a substantially straight shaft with a first end and a second end, the first end for removable attachment to the bone anchors;
an alignment tube removably and operatively attached to the second end of the at least two docking post, the alignment tube of a predetermined length relative to the docking post; and
a locator, the locator attached to an end of the alignment tube whereby the alignment tube is aligned with the first end of a first docking post, the locator including a lumen for accepting and guiding a surgical instrument to the first bone anchor.

12. The apparatus of claim 11 wherein the bone anchor is a screw.

13. The apparatus of claim 11 wherein the locator is curved.

14. The apparatus of claim 11 wherein the docking posts further comprise a locking device for locking the docking post to the bone anchor at a pre-selected orientation.

15. The apparatus of claim 14 wherein the alignment tube further comprises a shaped fitting such that the docking post aligns the bone anchors at a desired orientation.

16. The apparatus of claim 11 wherein the locator includes a continually changing radius.

17. The apparatus of claim 11 wherein the alignment tube includes a shaped connection fitting for insuring the proper orientation of the bone anchors.

18. An apparatus for attaching a therapeutic device between a set of pedicle screws inserted into the spine of a patient, comprising;
two or more docking posts, each docking post being a substantially straight shaft with a second end and a first end and including a lumen through the shaft, the first end of the docking posts removably attachable to the pedicle screws inserted into the patient, the distal end including means for mating with a bore in each of the pedicle screws, the docking posts further including markings for aid in aligning the bores of the pedicle screws such that the therapeutic devices are inserted through the lumen of the docking posts and be secured between the pedicle screws.

19. The apparatus of claim 18 wherein the bone anchor includes a bore through which the therapeutic devices are inserted.

20. The apparatus of claim 18 wherein the therapeutic device may be one or more of the group consisting of bars, wires, cords, and curable plastics.

21. A method of inserting a therapeutic device to a pedicle screw comprising:
inserting one or more docking posts into a patient;
attaching the one or more docking posts to a corresponding number of pedicle screws;
orienting the docking posts in a predetermined orientation;
mounting a crosspiece to a first end of the one or more docking posts;
fixing a locator to the crosspiece;
moving a surgical instrument through the locator to the pedicle screws and placing the therapeutic device in the desired location.
